(19) United States
(12) Patent Application Publication
(10) Pub. No.: US 2019/0041356 A1
(43) Pub. Date: Feb. 7, 2019

(54) ENHANCED GRAPHITE BASED ELECTRODE AND METHODS USING THE SAME

(71) Applicant: McMaster University, Hamilton (CA)

(72) Inventors: Si PAN, Burlington (CA); Raja GHOSH, Dundas (CA); M. Jamal DEEN, Dundas (CA)

(21) Appl. No.: 16/104,323

(22) Filed: Aug. 17, 2018

Related U.S. Application Data
(63) Continuation-in-part of application No. 15/749,232, filed on Jan. 31, 2018.
(60) Provisional application No. 62/200,736, filed on Aug. 4, 2015.

(51) Int. Cl. G01N 27/416; G01N 33/18; G01N 27/30

(52) U.S. Cl. CPC G01N 27/4168; G01N 27/308; G01N 27/301; G01N 33/182

(57) ABSTRACT

Systems, methods and devices relating to measuring free chlorine in liquid samples. An enhanced graphite electrode and sensors comprising the same are provided. The enhanced graphite electrode in conjunction with a reference electrode and a counter electrode can be used in a chronoamperometry mode to detect concentrations of free chlorine in liquid samples, especially static liquid samples. The enhanced graphite electrode can also be used in conjunction with a counter/reference electrode in a pulsed amperometric detection mode to detect concentrations of free chlorine in liquid samples, especially static liquid samples.

### RELATED APPLICATIONS

This application is a Continuation-in-Part of U.S. patent application Ser. No. 15/749,232 filed Jan. 31, 2018 which is a 371 of PCT/CA2016/050914 filed Aug. 4, 2016 which claims the benefit of U.S. Provisional Patent Application No. 62/200,736 filed Aug. 4, 2015.

### TECHNICAL FIELD

The present invention relates to systems and methods for measuring constituents in liquid samples. More specifically, the present invention relates to enhanced electrodes and methods for measuring free chlorine in water.

### BACKGROUND

Chlorine is widely used as a disinfectant in the water treatment industry for inactivation of pathogenic microorganisms such as Cryptosporidium and Escherichia coli. Before chlorine treated water can be sent from the treatment plant into the distribution system, it must meet certain standards for residual free chlorine concentration, which is typically below the 5 ppm range. Free chlorine content in municipal water is currently measured using N,N′-diethyl-p-phenylenediamine (DPD) based colorimetry. Other methods include amperometry techniques, in which the water is passed through a set of charged electrodes and the presence of free chlorine causes a signal change. There have been some efforts towards developing alternative detection methods, and improving or miniaturizing existing devices and methods. With increasing public awareness on water quality and tighter public health regulations and practices, such as point-of-use sampling and analysis, a robust, reliable, low-cost, and portable free chlorine sensor would be highly desirable. This is particularly relevant in small and remote communities, where highly-trained personnel may not be available, and routine maintenance is less feasible.

Several promising materials for free chlorine sensing using amperometry with linear response have recently been reported in the literature. However, the sensing materials are either expensive (e.g. glassy carbon, gold, boron-doped diamond, graphene, carbon nanotubes, ferrocene), or potentially leach hazardous materials (e.g. benzethonium chloride, aniline oligomers). Moreover, in most cases, the upper range for sensing was 2.0 ppm, and hysteresis during repeated measurements was not systematically studied. In typical water-testing applications, the concentration of free chlorine in the tested sample is likely to fluctuate and hysteresis, if present, would affect sensor performance. Equally important is the selectivity of the sensor, i.e. its ability to distinguish free chlorine from total chlorine, the latter being the combination of free chlorine and reduced chlorine in the form of chloride ions.

Attempts have been made to develop a free chlorine sensor that avoids the shortcomings of the prior art while addressing the needs of ease of use and suitability for rough, non-laboratory conditions. However, for most sensors, the flow of liquid passing through the electrodes needs to be regulated within a certain range for accuracy of measurement. Therefore, the requirement for constant and precise mixing of the liquid sample during chlorine measurement is a limiting factor since such sensors are not suitable for dip measurements, for example. Moreover, the detection of chlorine free (0 ppm) liquid samples is not reliable in existing sensors.

Therefore, there remains the need for improved sensors and more efficient and versatile methods for measuring free chlorine concentration in liquid samples.

### SUMMARY

The present invention provides an enhanced graphite working electrode for measuring level of free chlorine in a liquid sample. The present invention also provides methods for measuring the level of free chlorine in liquid samples using an enhanced graphite working electrode.

In a first aspect, the present invention provides an electrode comprising:

at least one section comprising modified graphite;

wherein

said electrode is for use in measuring a level of chlorine in a liquid sample;

said modified graphite is modified by a process comprising:
immersing graphite in an electrolyte solution with said graphite operating as a working electrode; and
applying a voltage to said graphite such that there is a voltage potential difference between said working electrode and a reference electrode of at least 0.8 V, and, at most, 1.15 V;

wherein
said electrolyte comprises ammonium carbamate prepared in a sodium phosphate buffer.

In a second aspect, the present invention provides a process for modifying graphite, the process comprising:

immersing said graphite in an electrolyte solution with said graphite operating as a working electrode; and

applying a voltage to said graphite such that there is a voltage potential difference between said working electrode and a reference electrode, said voltage difference being at least 0.8 V and, at most, 1.15 V;

wherein a resulting modified graphite is used in an electrode for measuring chlorine in a liquid sample.

In another aspect, the present invention provides a method for measuring a level of chlorine, the method comprising:

a) providing a sensor system in a liquid sample, said sensor system comprises a working electrode, a reference electrode and a counter electrode, said working electrode comprising:
at least one section comprising modified graphite; wherein said modified graphite is modified by a process comprising:
   immersing graphite in an electrolyte solution; and
   applying a voltage to said graphite such that there is a voltage potential difference between said graphite and a modification reference electrode of at least 0.8 V, and, at most, 1.15 V; and wherein
   said electrolyte comprises ammonium carbamate prepared in a sodium phosphate buffer;

b) applying a constant voltage between the working electrode and the reference electrode;

c) measuring a current of the working electrode over time during application of the constant voltage; and

d) correlating the current to the level of chlorine in the liquid sample.

In a further aspect, the present invention provides a method for measuring a level of chlorine, the method comprising:

a) providing a sensor system in a liquid sample, said sensor system comprises a working electrode and a reference electrode, said working electrode comprising:
at least one section comprising modified graphite; wherein said modified graphite is modified by a process comprising:
   immersing graphite in an electrolyte solution; and
   applying a voltage to said graphite such that there is a voltage potential difference between said graphite and a modification reference electrode of at least 0.8 V, and, at most, 1.15 V; and wherein
   said electrolyte comprises ammonium carbamate prepared in a sodium phosphate buffer;

b) applying a voltage between the working electrode and the reference electrode in the form of a plurality of pulsations of a fixed duration and at a fixed interval between each pair of said plurality of pulsations;

c) measuring a current of the working electrode at an end of each of said plurality of pulsations;

d) correlating the current to the level of chlorine in the liquid sample.

### BRIEF DESCRIPTION OF THE DRAWINGS

The present invention will now be described by reference to the following figures, in which identical reference numerals refer to identical elements and in which:

FIG. 1A is an illustration of a three-electrode experimental setup for modifying graphite according to one aspect of the invention;

FIG. 1B is a graph showing the transient current profile of measurements of free chlorine concentration of 0, 1, 3 and 5 ppm in a three-electrode chronoamperometry setup according to one aspect of the invention;

FIG. 2 is a graph showing data from FIG. 1B as a function of t^{-0.5} as present in the Cottrell equation according to one aspect of the invention;

FIG. 3 is a plot of the intercept and slope for the 4 repeated experiments shown in FIG. 1B according to one aspect of the invention;

FIG. 4A is an illustration of a two-electrode experimental setup for conducting pulsed amperometry detection (PAD) according to one aspect of the invention;

FIG. 4B is a schematic diagram of the finite difference method calculation of concentration during PAD according to one aspect of the invention;

FIG. 5 is a graph showing a PAD calibration curve for five repeated measurements at 0-5 ppm according to one aspect of the invention;

FIG. 6 is a graph showing a sample PAD signal during the measurement according to one aspect of the invention;

FIG. 7 is a graph showing a sample two-time constant fitting of charging current on a double log scale to expose deviations according to one aspect of the invention;

FIG. 8 is a graph showing the correlation of time constants, τ₁, τ₂ and the Debye length, κ⁻¹ according to one aspect of the invention;

FIG. 9 is a graph showing a comparison of concentration evolutions at x=0 during PAD according to one aspect of the invention;

FIG. 10 is a graph showing a comparison of concentration profiles at the end of PAD according to one aspect of the invention;

FIG. 11 is a graph showing an experimental data fit to first-order reaction kinetics with a DC component according to one aspect of the invention;

FIG. 12 is a representation over time of an artificial stimulation signal for PAD according to one aspect of the invention;

FIG. 13 is a circuit diagram of an implementation of a PAD instrument according to one aspect of the invention;

FIG. 14 is a graph showing a sample PAD signal depicted in the context of the raw signal according to one aspect of the invention;

FIG. 15 is a graph showing a PAD signal of a custom instrument, measuring a dummy cell of two capacitors in series according to one aspect of the invention;

FIG. 16 is a graph showing a PAD signal of a commercial instrument, measuring the same dummy cell as in FIG. 15 according to one aspect of the invention;

FIG. 17 is a graph showing a comparison of the custom and commercial PAD devices using the respective signals according to one aspect of the invention; and

FIG. 18 is a graph showing a surface plot of the PAD voltage (ΔV) as a function of the values of R₂ and R₃, each from 1 kΩ up to 1 MΩ according to one aspect of the invention.

In the drawings, embodiments of the invention are illustrated by way of example. It is to be expressly understood that the description and drawings are only for the purpose of illustration and as an aid to understanding, and are not intended as a definition of the limits of the invention.

### DETAILED DESCRIPTION

As noted above, there is a need for a free chlorine measurement system and method that is inexpensive, easy to use, applicable to non-laboratory conditions, and has greater accuracy, versatility and sensitivity. The present invention provides for enhanced working electrodes for use in measuring free chlorine in a static liquid sample. The present invention also provides simplified methods with increased limits of detection for measuring free chlorine in static liquid samples.

One embodiment of the present invention employs ammonium carbamate to electrochemically modify common graphite to fabricate a graphite-based electrode for sensing free chlorine in water samples, as disclosed in co-pending application U.S. Ser. No. 15/749,232. The contents of this co-pending application are hereby incorporated herein by reference. However, the invention disclosed by this previous application has been improved upon.

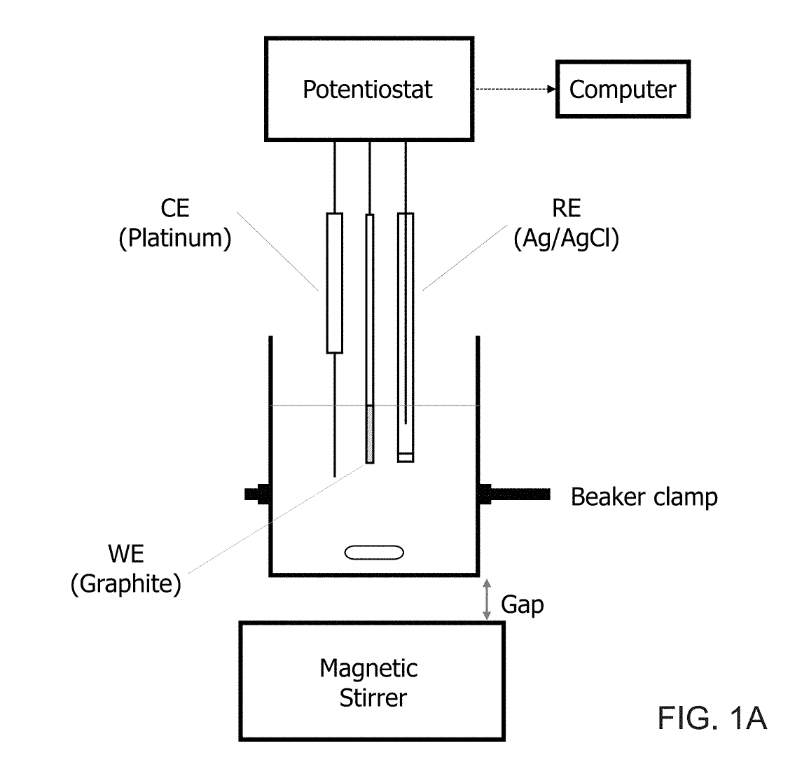

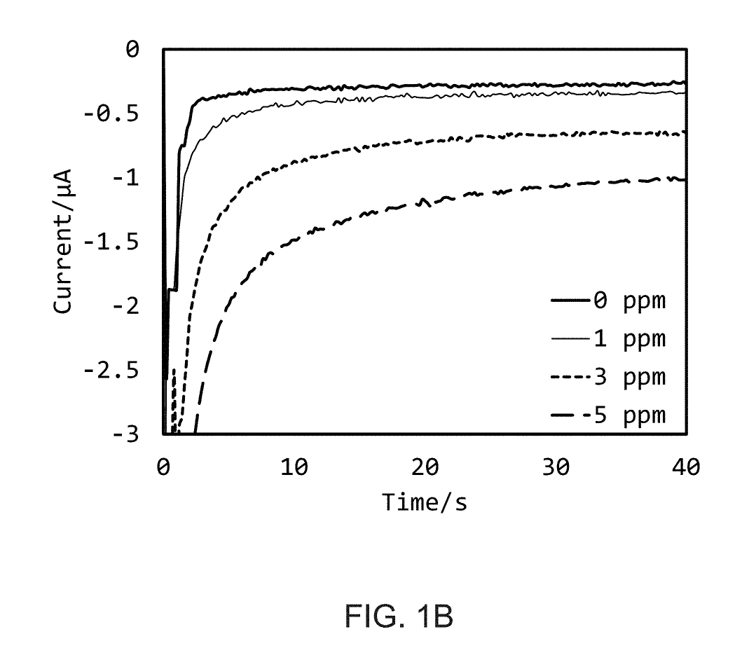

Specifically, the electrochemical modification of common graphite may be carried out in a three-electrode mode, as shown in FIG. 1A. Pencil lead was cleaned using lab tissue and rinsed with deionized water. The rinsed lead was then immersed in an electrolyte solution consisting of 0.1 M ammonium carbamate (292834-25 G) prepared in 0.1 M sodium phosphate buffer (pH 7.0), mixed until the pH reached 8.9. While immersed in the electrolyte solution, a voltage was applied to the lead, the voltage being 1.1 V versus a Ag/AgCl reference electrode similarly immersed in the electrolyte solution. An auxiliary (or counter) platinum electrode may also be used as a third electrode. In one experiment, the voltage (a potential of 1.1 V between the graphite working electrode and the reference electrode) was applied for approximately 7200 seconds. Regarding the temperature of the set-up, experiments have shown that a room temperature of between 19-24 degrees C. is preferred.

The voltage used in the above electrochemical modification may be from 0.8 V to 1.15 V. However, it is thought that using higher activation energy, in the form of a higher voltage, during electrochemical modification of the graphite may result in denser modification of the material. Preferably, the voltage may be from 1.1 V to 1.15 V, and most preferably 1.1 V. It was found that higher voltage did not produce increased gas evolution on the working electrode and the reaction is possibly accelerated to lead to higher intermediate production, and consequently denser graphite material in the form of an enhanced graphite working electrode. The enhanced graphite working electrode possesses improved properties (such as higher sensitivity) and have resulted in the development of advanced methods for free chlorine measurement, as described below.

In one embodiment, the enhanced graphite working electrode produced may be used for free chlorine sensing by chronoamperometry at a voltage of 0.1 V versus Ag/AgCl reference electrode using the above three-electrode setup. However, the process is conducted on unmixed or static liquid samples and analyzing transient current data. The fact that no mixing or stirring is required while conducting measurement is advantageous from a point of view of versatility and convenience, and a direct result of the improved properties of the enhanced graphite working electrode.

It will be appreciated that the above conditions are exemplary and other conditions can be used for the electrochemical modification, as would be appreciated by a skilled person in the art.

FIG. 1B shows the transient signal from the static sensing of free chlorine, using a three-electrode setup in a chronoamperometry mode. The setup using the enhanced graphite working electrode of the present invention is similar to that in International Patent Application WO2017/020133, however mixing of the liquid sample was not conducted. Briefly, the working electrode is used with a reference electrode, which is of a Ag/AgCl type with 1.0 M KCl as the filling electrolyte. The counter electrode is Pt wire. A voltage of 0.1V between the working electrode and the reference electrode is applied to the working electrode. The electrodes remained in the test solution for one minute before the measurement was initiated.

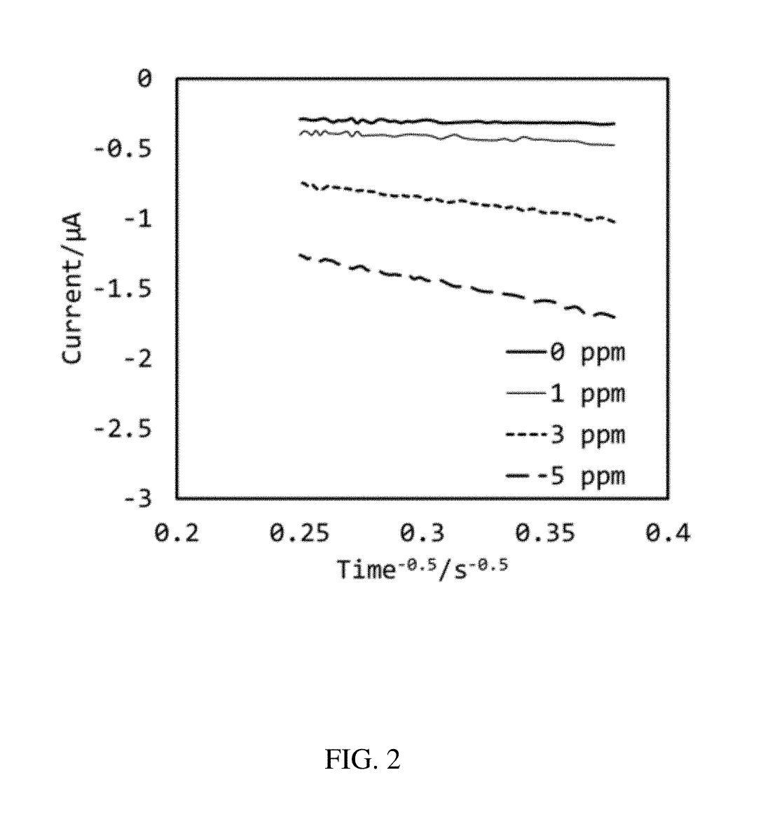

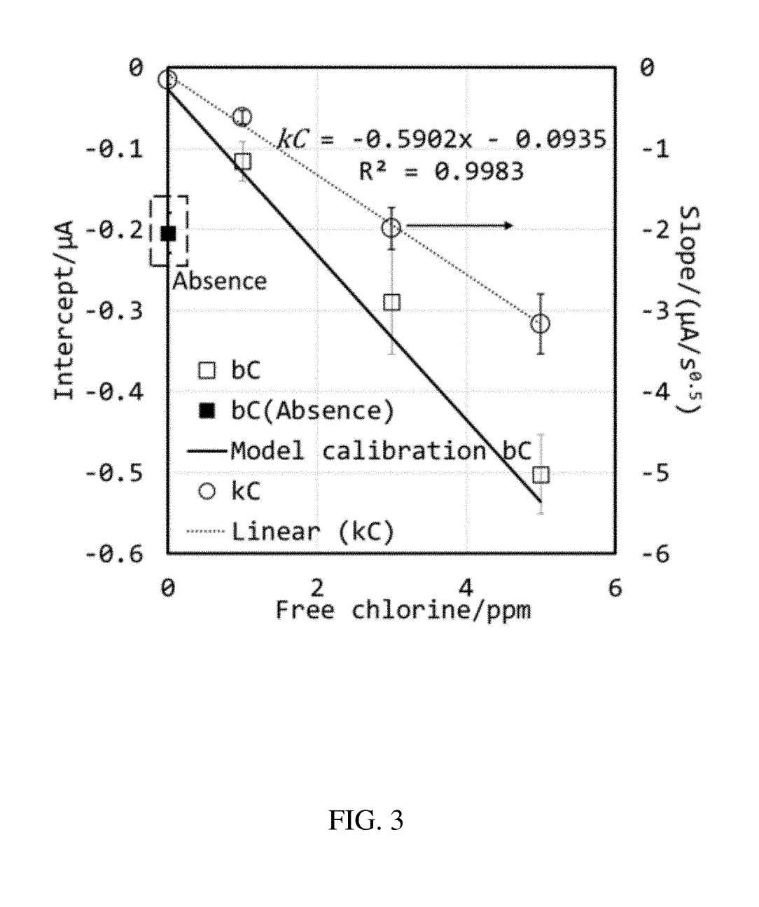

Transient current profiles of measurements of free chlorine concentration including 0, 1, 3 and 5 ppm where completed (FIG. 1B), compared, and further investigated for information from these profiles (FIGS. 2 and 3). Using an expanded Cottrell equation and taking into consideration the geometry of the sensor, calculations were conducted to effectively determine free chlorine measurement in static liquid samples.

To perform calculations and experiments around the expanded Cottrell equation, an electronic circuit was designed along with a corresponding method for carrying out the relevant calculations. The current value measured every 500 ms was recorded in a Static Random Access Memory (SRAM) of a microcontroller. The time data underwent conversion calculations based on the Cottrell equation, as shown in FIG. 2. The slope of the Cottrell equation was then fitted using a linear least square approach and the intercept was calculated based on the slope, the means of the converted time, and the measured current (see FIG. 3). The findings and details of the calculations are explained below in the Examples section.

Based on these results, further studies have led to the development of a pulsed amperometric detection (PAD)-based method for measuring free chlorine. The PAD-based method involves the application of a pulsed voltage for a short period of time, while measuring the current at the end of each pulse, while the above mentioned chronoamperometry method applies a constant voltage and records the current profile over time. The advantages of this PAD method, apart from being applicable to a static liquid sample, may include the lack of a need for regular replacement of the probe solution, the absence of a dedicated reference electrode, and the ability to detect the absence of free chlorine (0 ppm) directly. Therefore, the use of the enhanced graphite working electrode has led to improved methods for measuring free chlorine in liquid samples.

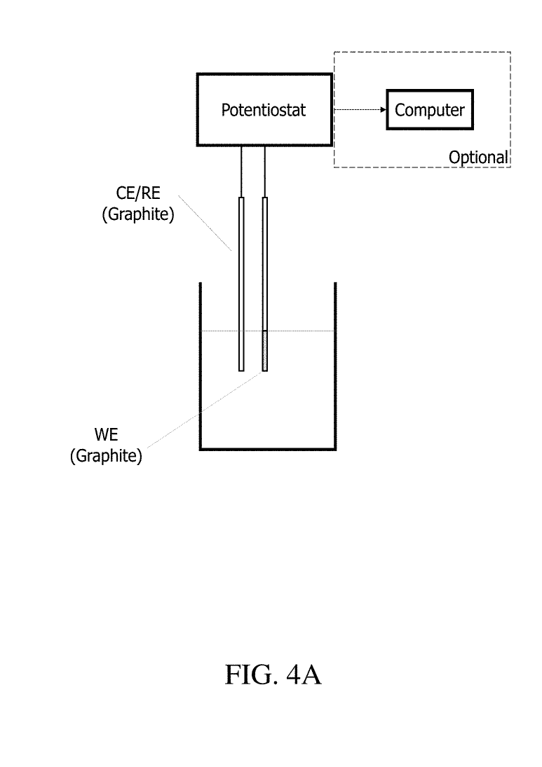

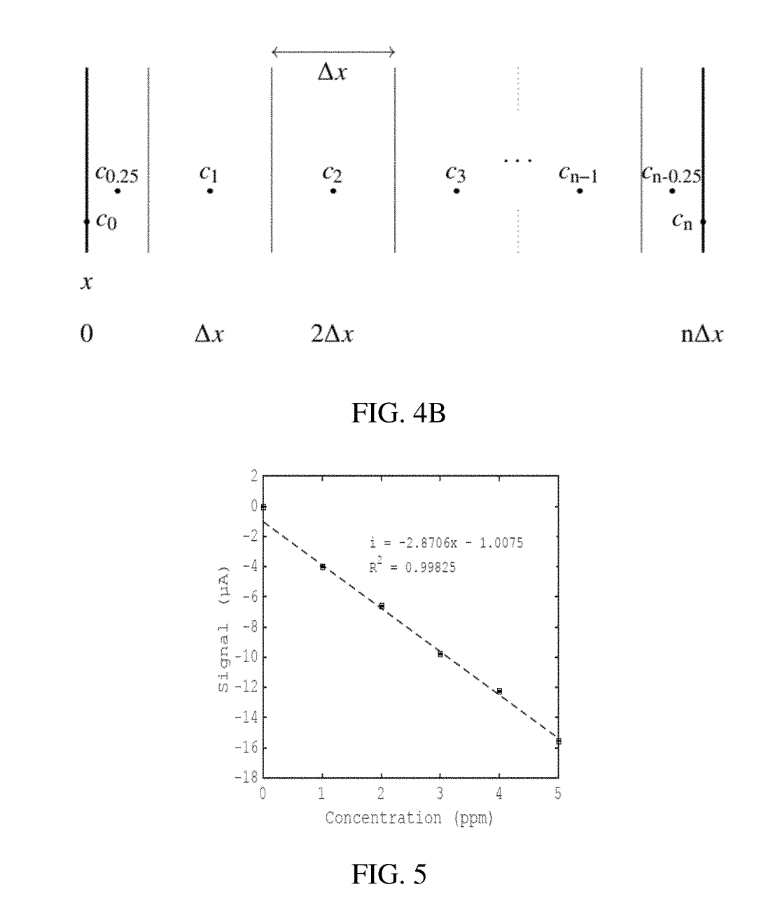

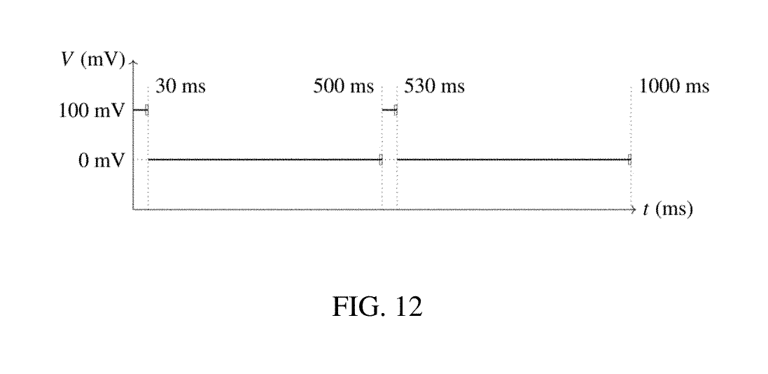

The PAD method comprises providing a sensor system in the liquid sample as exemplified in FIG. 4A. The sensor system may comprise the enhanced graphite working electrode and a reference/counter electrode (two-electrode system). Preferably, the reference/counter electrode is an electrode having an open-circuit potential similar to that of the enhanced graphite working electrode. In one embodiment, the reference/counter electrode is an unmodified graphite electrode. The method further comprises applying a pulsed voltage at predefined intervals. For example, the voltage could be applied for 30 ms at 500 ms intervals as shown in FIG. 12, but acceptable intervals may be, for example, between 120 ms to 120,000 ms. The duration of each pulse may be from 15 ms to 60,000 ms, preferably 30 ms. In one preferred embodiment, the interval between each pair of pulses is at least four times the duration of the pulse.

The current is measured at the end of each pulse. To obtain a measure of the level of chlorine in a liquid sample, correlation between the signal difference and the concentration of free chlorine is made according to calculations explained below. The level of chlorine that could be detected may be from 0 ppm to up to 20 ppm, preferably from 0 ppm to 5 ppm. Preferably, the liquid sample is a static liquid sample that does not require mixing or stirring during the measurement process. In addition, such a two-electrode system does not require a separate dedicated reference electrode. A dedicated reference electrode needs regular maintenance to replace the electrode solution and, in addition, using a dedicated reference electrode requires the electrode solution to be at a constant concentration. However, it is preferred that there be no liquid components in the sensor systems.

In one exemplary experiment, an EmStat 3 (PalmSens, the Netherlands) was set in PAD mode with the following parameters: tᵢₙₜₑᵣᵥₐₗ=0.5 s, tₚᵤₗₛₑ=0.03 s, Eₚᵤₗₛₑ=0.1 V, E_{dc}=0.0 V and t=15 s. The iₚᵤₗₛₑ was reported as the raw signal of the ADC. The enhanced graphite working electrode produced as described above was used as the working electrode and an unmodified pencil lead polished with lab tissue was used as the reference/counter electrode. The testing solution was 0.1 M pH 7.0 sodium phosphate buffer with free chlorine from 0 to 5 ppm. The two electrodes were allowed to equilibrate for 45 seconds. The charging response current was measured using a two-electrode setup by connecting the counter and reference terminals of EmStat 3 together. For each concentration, 10 cycles of charging and discharging were carried out consecutively and each concentration was repeated 5 times. The electrodes were dried by air between each repeated measurement.

It should be noted that the PAD method may be used with a three-electrode setup as described above for a chronoamperometry chlorine measuring system. However, the additional cost of a third electrode and additional electronic components, while having no sensitivity benefits, would suggest that a two-electrode setup is preferred. It should also be appreciated that the PAD method may be conducted on a mixed sample for measuring free chlorine in the sample. Again, the sensitivity would not be affected.

As will be explained in the Examples, use of the PAD method to detect free chlorine is dependent on ionic strength and thus requires that the conductivity of the solution be known. Any known method in the art may be used to determine the conductivity of the liquid sample. A conductivity meter used to measure the conductivity of the sample may be separate from the sensor system or may be incorporated in the sensor system of the present invention.

Practically, the PAD method is an improved method for measuring free chlorine because, although the conductivity meter may be a separate instrument or circuit on a circuit board, such a conductivity meter requires much less maintenance than a dedicated reference electrode (three-electrode system). As is known to those with of skill in the art, a conductivity meter requires no regular maintenance.

Finally, once the calculations of both the Cottrell equation experiment and the PAD method are complete, the fitted signal can be provided to output devices for display to a user. Such displays and other output means can include seven-segment number displays, pixel-matrix displays, and serial communication protocols for smart devices or personal computers.

### EXAMPLES

As mentioned above, transient current profiles of measurements of free chlorine concentrations including 0, 1, 3 and 5 ppm were completed in a three-electrode chronoamperometry mode, compared, and further investigated for information from these profiles. The transient signal was replotted using the Cottrell equation, as a function of t^{-0.5}:

i=nFAD^{0.5}c(πt)^{-0.5} (1)

In the above equation, n is the number of electrons transferred, F is the Faraday constant, A is the area of the electrode, D is the diffusion coefficient, c is the bulk concentration of the analyte, and t is the time. The reaction was assumed to be much faster than mass transport, i.e. diffusion.

The classic Cottrell equation considers unsteady state diffusion at a semi-infinite plane. For any other geometry, an expansion term is added, e.g. specifically for a sphere, the expanded Cottrell equation is:

i=nFAD^{0.5}c(πt)^{-0.5}+nFADcr⁻¹ (2)

where r is the radius. The ratio of the slope and intercept for a sphere is equal to a term independent of c, or t. For a cylinder, obtaining an analytical solution is difficult and hence a numerical approximation involving Bessel functions is used. This makes it incompatible with low-cost electronics and explains why a new method to determine concentration was needed.

FIG. 2 shows the current data obtained between 7-16 s plotted against t^{-0.5}, consistent with the Cottrell equation. The time slice selected is the typical range for faradaic dominant systems. The resulting straight lines obtained at the different free chlorine concentrations are typical of those obtained during unsteady state diffusion-based mass transfer. The slope of the straight lines increased with free chlorine concentration.

Conforming with published results for diffusion-controlled mass transfer, the profiles were straight lines in the relevant time window. The slopes increased with the free chlorine concentration, while for 0 ppm, it was nearly a flat line. The irregular 0-ppm profile spacing is inherited from FIG. 1C and is further illustrated in FIG. 3.

FIG. 3 shows plots of slope and intercept data obtained from four repeated experiments as functions of free chlorine concentrations. These results suggest that both slope and intercept correlated linearly with concentration, and could therefore be used as tools for concentration measurement. The respective values of b and k are −0.1057 μA ppm⁻¹ and −0.5902 μA s^{-0.5} ppm⁻¹.

The calibrated (bc) and (kc) were −0.1018 μA ppm⁻¹ and −0.5902 μA s^{-0.5} ppm⁻¹, respectively. The intercept of 0 ppm was an outlier, indicating the absence of free chlorine. This is due to the theoretical difference between the charging and faradaic currents. The slopes were linear for the entire range to use as a concentration calibration.

The geometry of the employed sensors may well be different from a semi-infinite plane. In these geometries, the mass transfer equations from solving Fick's Laws will have expansion terms. Based on published literature, the geometry of a semi-infinite plane can be used for systems where the diffusion length is much smaller compared to the cylinder's diameter. However, in the present system, the diffusion layer surrounding the electrode would be approximately 200 μm while the diameter of the working electrode is 700 μm. Therefore, when using a planar approximation for the present system, an expansion term for the transient current should be included to form the general form of the expanded Cottrell equation:

i=kct^{-0.5}+bc (3)

where k is the lumped term (nF AD^{0.5}π^{-0.5}) and b needs to be experimentally determined for each specific setup due to the non-standard geometry in question.

To establish whether this correlation could be used for calibration, a new set of experiments were carried out and the data obtained was replotted according to this equation. The respective average values for intercepts (bc) and slopes (kc) thus obtained were plotted as shown in FIG. 3. While the slope and intercepts of the free chlorine-samples fit the model, free chlorine-free samples did not. In the absence of free chlorine, a charging current response at the solution interface could be expected, whereas in the presence of free chlorine, additionally the Cottrell equation-based response would be expected. Therefore, the sensor is not only suitable for measuring free chlorine concentration but also very useful for certifying free chlorine-free samples, as required with chlorine filters and in the bottling industry.

FIG. 4B shows a standard discretization of space at the surface of the electrode for the finite difference method. This method is used to calculate and illustrate the concentration change during pulsed reaction in contrast with that during a constant reaction. The boundary condition at the electrode surface is expressed in a first order reaction kinetics, as in the following equation:

(c_{0,t+Δt}−c_{0,t})/Δt=−kc_{0,t} (4)

Assuming that each space element in the concentration is uniform, this boundary condition couples the reaction and diffusion during a pulsed reaction.

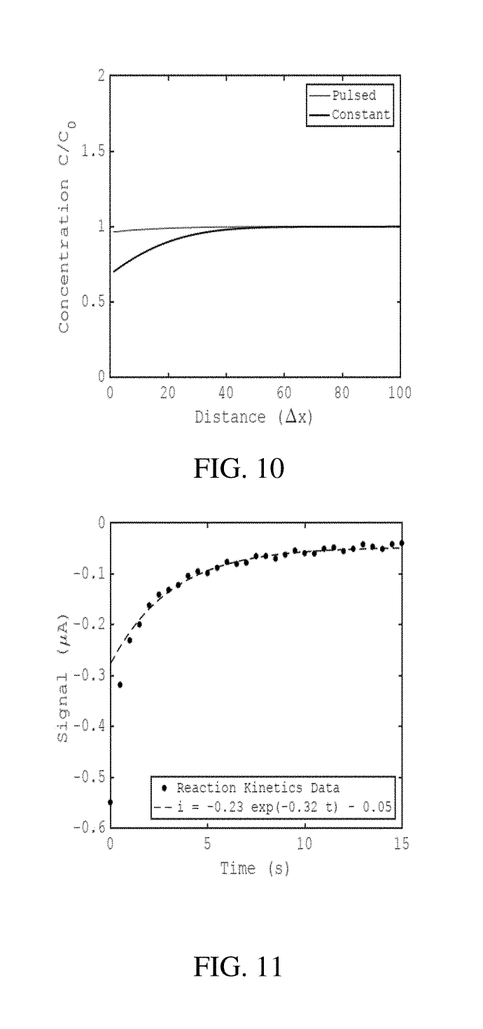

The contrast of concentration change at the surface is shown as FIG. 9. The concentration profile at the end of the simulation is shown as FIG. 10.

FIG. 5 shows a calibration curve of the PAD signal using a two-electrode system (working electrode and counter/reference electrode). The correlation was linear. The error bars are propagated errors from all the measurements. The zero-free chlorine measurement falls off the calibration curve in the opposite direction of the signals, indicating that the absence of free chlorine has been reliably detected.

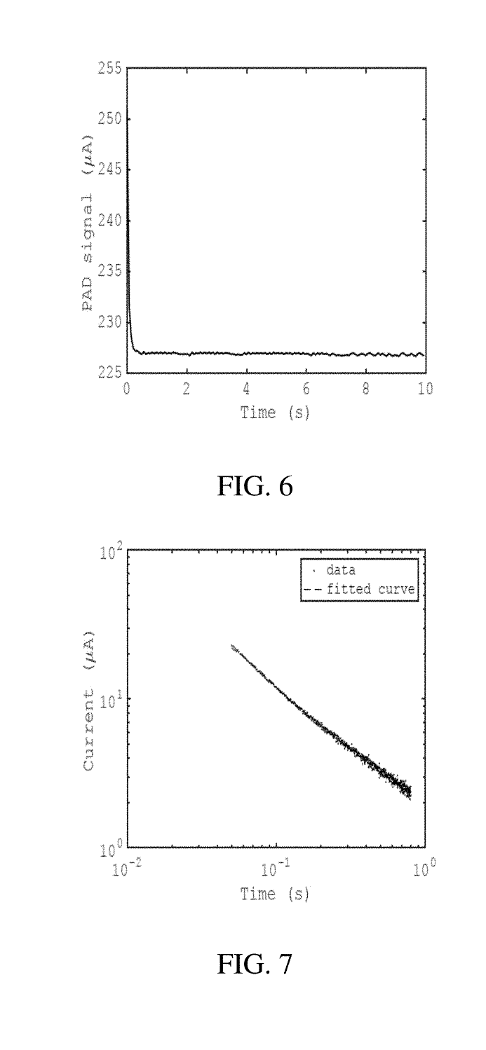

FIG. 6 examines the signal of a PAD experiment, showing an initial steep decrease followed by a relatively long period of stable signal. The initial steep decrease is due to the charging current attaining equilibrium, while the relative plateau comprises the pulsed reaction under equilibrium of the charging current.

Attaining a dynamical equilibrium under the repeated pulsed voltage is the essence of the PAD approach. For a given setup, with all the factors fixed, the primary current depends on the time delay after the applied voltage, and the secondary current depends on this time delay and bulk concentration. If a current measurement is taken at a fixed time delay, it is only directly proportional to the bulk concentration. The principle can be expressed in the following equation:

_{PAD}=i_{c}(t)+iᵣ(t,c) (5)

where is is the charging current (primary) and iᵣ the reaction (secondary) current. Measuring the current at a fixed time delay, repeatedly at fixed intervals, is the essence of a PAD method. As suggested in FIG. 9, the concentration profile over time during PAD is considered much more stable compared to that in chronoamperometry.

FIG. 7 shows a sample fit of a two-time constant function to the charging current on a double log scale. The fitted curve overlaps with the measured data points. The curve can roughly be seen as two sections of a straight line. Towards the longer time, the data points have increased noise levels.

The fit suggests the existence of two capacitances during the charging current. Both capacitances depend on the respective time constant. The total charging current for a given system depends on the time at which the current is measured.

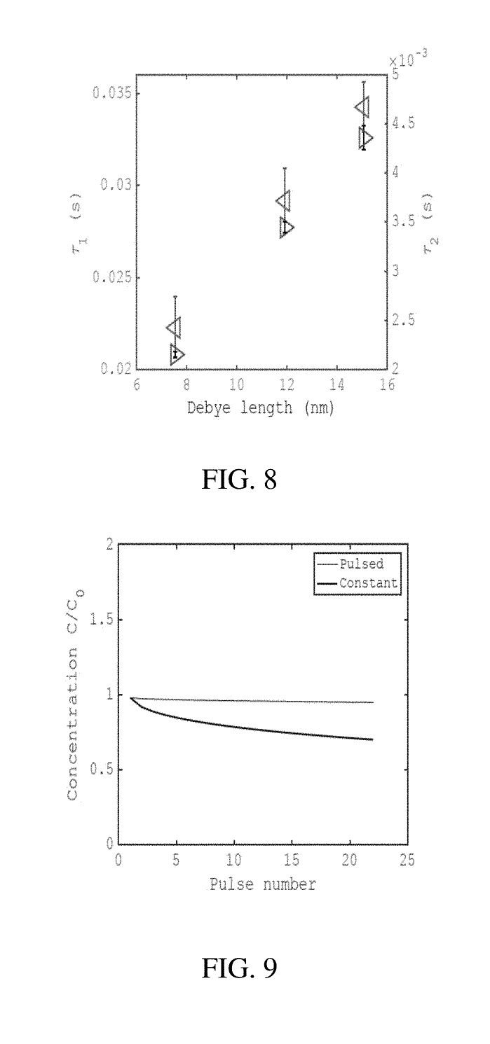

FIG. 8 shows the correlation of time constants and the Debye length. The Debye length in solution is a measure of a charge carrier's net electrostatic effect and how far its electrostatic effect persists. There is a linear trend between the time constants and the Debye length, both of which are dependent on the ionic strength (I) of the solution. τ₁ was approximately one order of magnitude higher than τ₂. For each data point, five repetitions, each consisting of 10 measurements, were carried out. As the dependence of τ on I is not linear, this figure uses τ⁻¹ as the independent variable to show the clear trend. The measurement using laboratory deionized water was discarded due to the square root-reciprocal relationship between κ⁻¹ and I. The triangles point to their respective axis.

In FIG. 8, it can be seen that the time constants depend on the Debye length τ⁻¹, which depends on the ionic strength I. This section discusses the derivation. A time constant τ for a capacitor is a product of the resistance R and the capacitance C(τ=RC). In lower concentrations (typically <0.2M) the conductivity is proportional to the ionic strength. The resistivity ρ, being the inverse of conductivity, depends on the ionic strength as follows:

ρ∝I⁻¹ (6)

For a given geometry, the resistance is proportional to the resistivity. The capacitance, on the other hand, depends on the inverse of the Debye length, which depends on the inverse of the square root of the ionic strength:

C∝κ

κ⁻¹∝I^{-0.5}

τ=RC∝I^{-1+0.5}=I^{-0.5} (7)

The time constant depends linearly on the Debye length, i.e, the inverse of the square root of the ionic strength. The intent of this figure (FIG. 8) is to show a clear linear dependence, therefore the time constants were plotted against the respective Debye lengths.

FIG. 9 shows, in the non-pulsed case, the concentration at the surface of the working electrode at each time point of data recording. The initial concentration was close to one. The concentration decreased towards a stable value, characterized by the decrease of change per time.

FIG. 9 also shows, in the pulsed case, the concentration at the surface of the electrode at each time point of data recording. The initial concentration was also close to one, the same as the non-pulsed case. The concentration stabilized to the same criterion much sooner using the criterion from the non-pulsed case. At any given time after the beginning, the concentration was higher in the pulsed cased than in the non-pulsed case. The final decrease in concentration was a few orders of magnitude lower in the pulsed case. Due to the relatively small deviation, the change in concentration in the case of PAD appears more stable. The profile from PAD may appear as a stable signal to instruments with limited sensitivity or resolution.

Diffusion and reaction work in opposite directions to change the concentration. For diffusion, a high concentration difference will lead to higher replenishment due to a higher rate of diffusion from bulk to the electrode-solution interface. With time, c₀ is in a decreasing trend whereas c_{bulk} remains unchanged. For reaction, a lower concentration will lead to a lower consumption rate. The reaction only happens at the surface, depending only on c₀. Higher replenishment and lower consumption will help maintain the surface concentration at a relative stable value.

FIG. 10 shows the final concentration profile of the non-pulsed case. In a typical reaction-diffusion scenario, the concentration at the reaction surface decreases over time due to consumption. The concentration profile develops a diminishing slope going away from the reaction surface. This was the case in the non-pulsed reaction simulation result in this figure.

FIG. 10 also shows the final concentration profile of the pulsed case. Different from the pulsed profile, the apparent diminishing concentration was not obvious under the same scale. This indicates that the reaction did not consume a significant amount of the analyte. It starts to appear indiscernible a short distance away from the interface.

During the calculation, the boundary condition at the electrode spatial unit requires a model of the reaction kinetics. It is used to calculate the concentration change as a result of the reaction. The diffusion coefficient was assumed to be the constant across all time and locations. The reaction was assumed to start and finish instantly when the voltage from PAD changes.

Some implications can be drawn from the results of the simulation. If analyzed closely, the onsets of the spatial concentration profiles were comparable, suggesting that the sensor geometry of both PAD and chronoamperometry should be spaced the same way when designing the electrode layout.

FIG. 11 shows experimental data fit to first-order reaction kinetics with a DC component. The magnitude of the current decreased over time. The first four points were not used for fitting, and disagreed with the fitted curve; they contained significant charging current. Despite some level of noise, the majority of the fitted curve overlapped with the experimental data points. This indicates an adequate fit to describe the reaction kinetics using the model. It is required by one boundary condition in the mass transfer calculations. The mass transfer calculation helped explain the stable signal during PAD as compared to during chronoamperometry.

The reaction kinetics were measured under vigorous mixing, assuming the mass transport does not limit the reaction. Four repeats were done to show the same trend with almost identical raw readings and fitted parameters. It also indicates that the free chlorine is being consumed. The successful fittings for the first order kinetics also help support the sensing mechanism of free chlorine consumption rather than adsorption equilibrium.

FIG. 12 shows an artificial stimulation signal for pulsed amperometry. The voltage V₁=0.1 V is applied for Δt₁=30 ms and V₂=0.0 V for Δt₂=470 ms, respectively. Usually, the signal is measured at the end of both periods of Δt₁ and Δt₂, marked using rectangles. A specific voltage is applied between the working electrode and counter electrode for m amount of time, followed by another voltage applied for n amount of time (usually n>>m). At the end of each period, the current is measured. Usually these periods are in milliseconds to seconds, and the shorter period is denoted as the pulse branch and the longer called the direct-current branch. The currents are called iₚᵤₗₛₑ, and i_{dc} respectively. Additionally, iₚᵤₗₛₑ-i_{dc} is called i_{diff}.

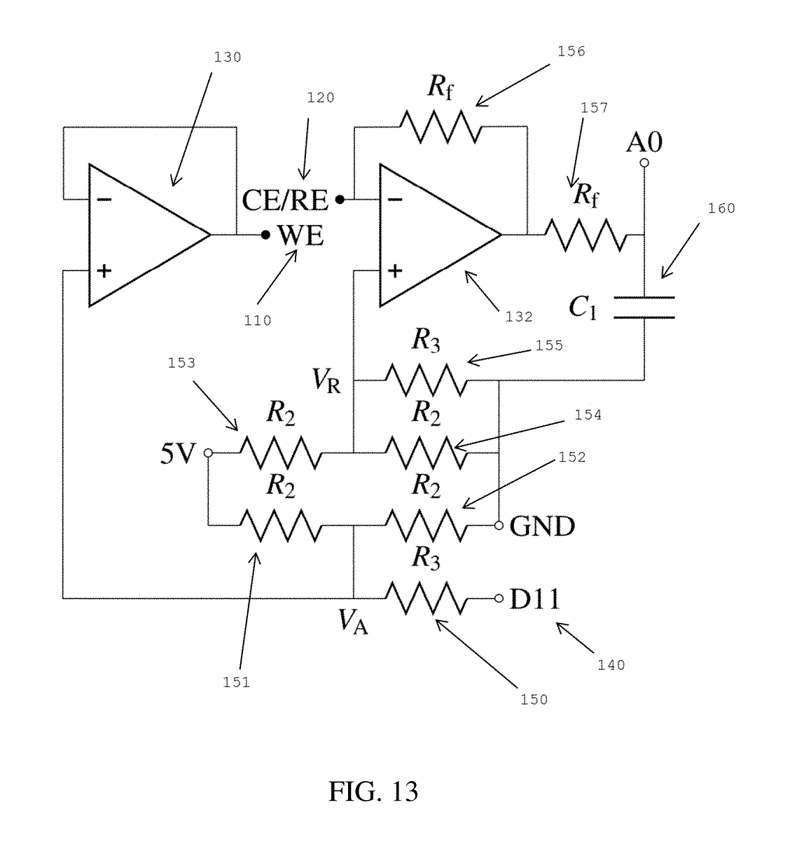

Referring to FIG. 13, a circuit diagram of an implementation of a pulsed amperometric detection instrument is illustrated. In this embodiment, a system 100 uses a working electrode 110 and a reference/counter electrode 120. The working electrode 110 and the reference/counter electrode 120 are immersed in a liquid sample to be tested (not shown). The working electrode 110 is connected to the output of a first operational amplifier 130 and the reference/counter electrode 120 is connected to the negative input of a second operational amplifier 132. The negative input of the first amplifier 130 is coupled to the working electrode 110. The positive input of the first amplifier 130 is coupled to a node V_{A}. Node V_{A} couples to a microcontroller node D11 140 through a resistor 150. Node V_{A} is also coupled to resistors 151 and 152. One end of resistor 151 is coupled to node V_{A} while the other end of resistor 151 is coupled, preferably, to a 5V power supply. One end of resistor 152 is coupled to ground while the other is coupled to node V_{A}. Resistor 153 is coupled between the 5V power supply and a node V_{R} while resistor 154 is coupled between node V_{R} and ground. Node V_{R} is coupled to the positive input of the second amplifier 132. Coupled between node V_{R} and one end of capacitor 160 is resistor 155 and this end of the capacitor 160 and resistor 155 are also coupled to ground. Between the negative input of the second amplifier 132 and the output of the second amplifier 132 is resistor 156. Between the output of the second amplifier 132 and a second end of capacitor 160 is resistor 157. The junction between the resistor 157 and capacitor 160 is coupled to node A0. Regarding the values of the various resistances, resistors 151, 152, 153 and 154 are preferably 20 kΩ (R₂) , while resistor 150 is preferably 500 kΩ (R₃). Resistors 156 and 157 are preferably 820 kΩ (R_{f}). It should be clear that node A0 is the input to a suitable ADC such that the output analog values of the second amplifier are converted into digital values. Preferably, the microcontroller is an Arduino/Genuino Uno using an ATmega 328 microcontroller.

The voltage at node V_{R} is measured to be 2.39 V, while at node V_{A,} V_{A,LOW} is 2.48 V, while V_{A,HIGH} is 2.48 V as measured by a multimeter. A direct measurement of ΔV when the signal at node D11 is HIGH or LOW is 93.8 mV and −0.7 mV, respectively. During the measurement, the microcontroller D11 is set either HIGH or LOW to allow adequate time for the multimeter to finish reading.

To implement the PAD method, the difference between V_{R} and V_{A,HIGH} in FIG. 13 is sought to be near 0.1 V. The resistors of the reference potential (V_{R}) has the following relationship as shown in Equations 8 and 9.

$$\begin{matrix}\frac{{V}_{c c}}{{R}_{2} + {R}_{p}} = \frac{{V}_{R}}{{R}_{p}} & ( 8 ) \\ where & \\ {R}_{p} = \frac{1}{\frac{1}{{R}_{2}} + \frac{1}{{R}_{3}}} & ( 9 )\end{matrix}$$

The resistors of the pulsed potential (V_{A}) work as follows: the D11 can output either of the two values: 0 V and 5 V. When V_{D11}=0 V, V_{cc}/(R₂+Rₚ)=V_{A}/Rₚ, and is identical to the fixed potential shown in Equation 8. The voltage applied to the working electrode is now 0 V. When V_{D11}=5 V, V_{cc}/(Rₚ+R₂)=V_{A}/R₂. The voltage applied to the working electrode is a non-zero value depending on the choice of R₂ and R_{3.} It should be noted that V_{R}=V_{A,LOW}, theoretically. When using R₂ (20 kΩ) and R₃ (500 kΩ) the resultant voltage between the two cases is theoretically either 0.980 V or 0 V. By varying the time periods during which V_{D11} is set to 0 V or 5 V, we can control the time periods of this simple PAD implementation. The voltage difference has to be re-adjusted by a different combination of resistors. There are advantages to this approach:
1) the mechanisms are simple (other than the setting of V_{D11}) and is discussed below;
2) the circuit building is straightforward as a result of 1); there is no need to use a voltage generating integrated circuit component;
3) programming V_{D11} between the two values is simpler than programming a voltage generating integrated circuit component (e.g. a digital-to-analog converter (DAC));
4) switching V_{D11} is faster by mechanism than switching a voltage generating integrated circuit component (e.g. DAC); and
5) six generic resistors usually cost less than a DAC.

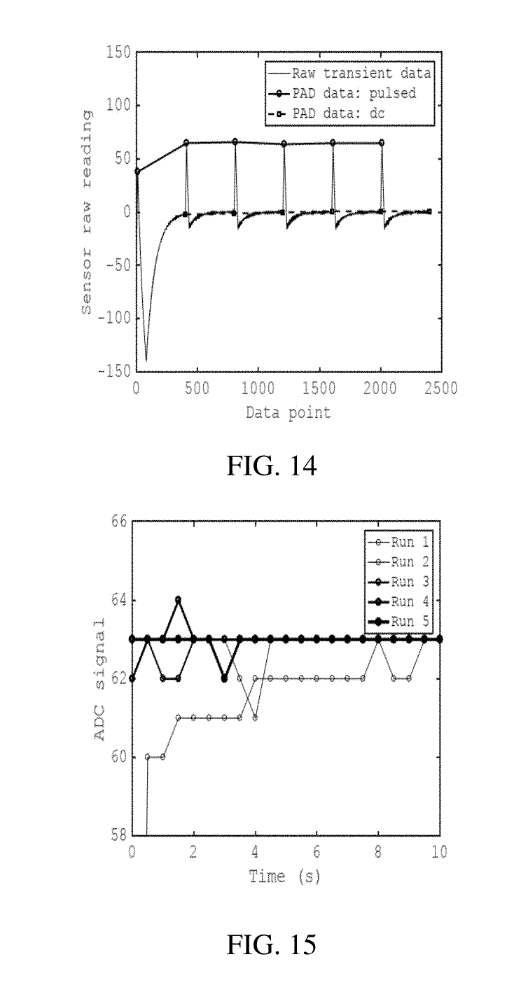

FIG. 14 shows the sampled data points in the context of the transient data using a dummy cell and the mechanism of PAD. The connected circles are the pulse samples of the PAD, iₚᵤₗₛₑ. Likewise, the dashed connected squares are the dc samples of the PAD i_{dc}. Due to the repeatability and stability shown in FIG. 14, the connected circles and the dash connected squares formed a stable signal after the initial period. Either iₚᵤₗₛₑ or i_{dc} or the difference between the two can be reported as the signal of PAD, usually by options in the instrument program. In subsequent measurements, the format of connected circles will be used as the PAD signal because the i_{dc} always reaches zero before the next pulse starts.

Similarly, the dashed connected squares are the dc samples of the PAD i_{dc}. Due to the repeatability and stability, the connected circles and the dash connected squares formed a stable signal after the initial period. Either iₚᵤₗₛₑ or i_{dc} or the difference between the two can be reported as the signal of PAD, usually by options in the instrument program. In subsequent measurements, the format of connected circles will be used as the PAD signal because i_{dc} reaches zero before the next pulse starts.

The PAD signal omits the detailed transient of the current, e.g. the downward peak near data point 100 in the figure. The implication is that if the sensor surface has been prepolarized, there could be a transient deviation of current in the first pulse. In some cases, this may cause an unexpected, unrecorded reaction. This should be kept in mind when using PAD for sensitive chemicals, such as those adsorbed on the electrode surface. Furthermore, in a PAD, it is also advised against the use of a preconditioning period by keeping the voltage at a certain value to let the electrode surface attain equilibrium. The pulsed nature means a PAD measurement is inherently transient, and the eventual equilibrium, if any, should be reached during the measurement. The preconditioning period is usually unrecorded.

FIG. 15 shows PAD signals of the custom instrument, measuring a dummy cell of two capacitors in series. Five repeated runs each gave stabilized readings. The first run was less stable compared to the rest. The smallest signal step was one ADC value.

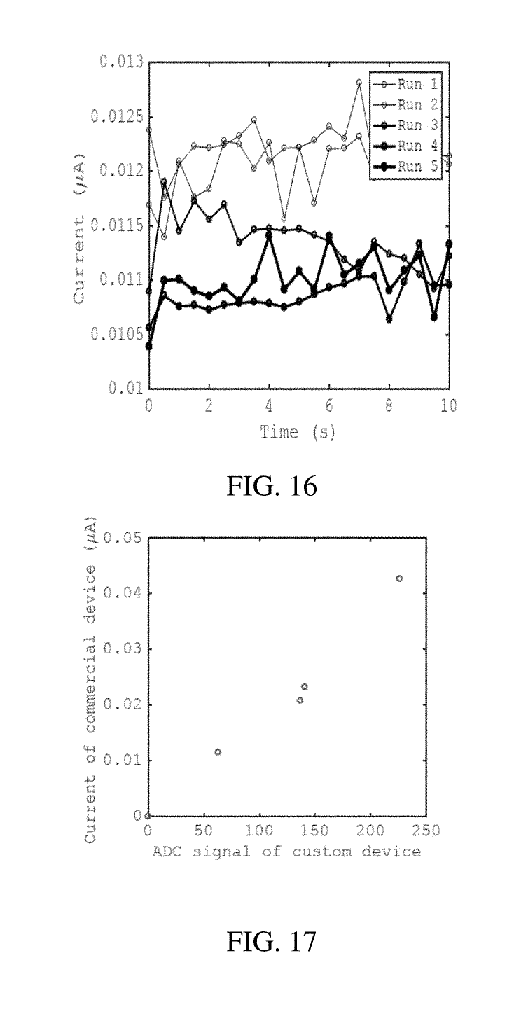

FIG. 16 shows PAD signals of a commercial instrument, measuring the same dummy cell. Five repeated runs each gave stabilized reading. The data points were more random in each run. The y-axis difference between each data point did not show clear steps.

FIGS. 15 and 16 show the PAD signal from a custom and a commercial device, respectively. The key features are the stabilizing trend and the intermittent data points at the set 500 ms intervals. In each measurement, the last 5 stable points were averaged as the reading of the measurement.

In FIGS. 15 and 16, both data sets from the in-house and commercial instrument show similar trends and features regarding the stabilization. The apparent noise is lower in the in-house data because the A0 can convert up to 1024 different values. Comparatively, the commercial instrument converts up to 65536 (2¹⁶ values). It is believed that the differences between the cases are large enough for the resolution of the implementation described above.

FIG. 17 shows a comparison of the custom and commercial PAD devices using the respective signals. The four different dummy cells measured suggests practical feasibility as there is an almost linear correlation between the signal measured by the commercial instrument and the signal measured by the in-house implementation. The raw ADC values were used for simplicity because in any real measurement, the reading, whether current or ADC values, shall be calibrated against another physical variable, such as concentration.

If further automation is needed the instrument may be allowed to integrate with other instruments to form a custom apparatus. By using a dedicated instrument, the operations of the experimenter can be minimized to reduce human error and to increase efficiency. However, the software routine has to be established using a comprehensive instrument first.

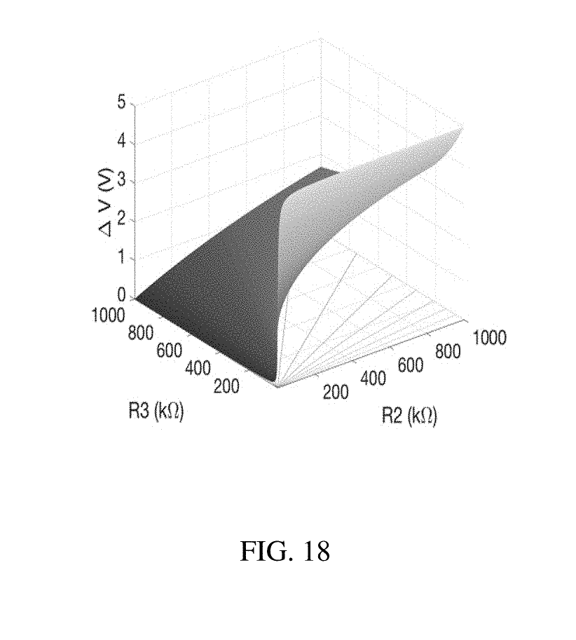

FIG. 18 shows the surface plot of the PAD voltage (ΔV) as a function of the values of R₂ and R₃, each from 1 kΩ up to 1 MΩ.

The ranges of R₂ and R₃ allow for virtually all required potential from ground (0 V) to V_{cc} (5 V in this case). The surface has no local minimum or maximum.

For a given PAD voltage, R₂ and R₃ are allowed to vary linearly with respect to each other, as depicted by the straight-line contour on the X-Y plane.

In FIG. 18, it is clearly not possible to find a local minimum or maximum to account for the variation of the resistor value from batch to batch. As a result, a high tolerance resistor may be used. In this design, both R₂ and R₃ had a tolerance of ≤1%.

The R₁ is omitted intentionally as a reminder that the voltage dividers can have more flexible choices. In a case where the three resistors can all vary freely, the calculation to assess the situation may be more complicated than practical.

Generating the voltages this way has advantages in practice. Firstly, the cost of six resistors is lower than using one integrated circuit to generate voltages. When the device is designed to be a dedicated system, there will be no further adjustment of the voltages, rendering the many options, such as a conventional DAC, uncompetitive. Secondly, the system is more efficient when compared with an added integrated circuit, as the added integrated circuit would require constant communication with the microcontroller. The size of the program used may also be larger, taking up the already limited space on the low-end microcontroller.

It should be noted that batch to batch variation of the resistors may require tweaking between the various resistor values. In the target region of intense blue, a small variation in R₂ requires a large change in R₃ in order to maintain ΔV near the target value. The precision of R₂ takes precedence over that of R₃.

An error between the linear fit function used in the microcontroller compared to that used in the iterative numerical method may arise. In some applications, this error will be included during the calibration step, after which the calculations will be carried out in the microcontroller.

While the present application has been described with reference to examples, it is to be understood that the scope of the claims should not be limited by the embodiments set forth in the examples, but should be given the broadest interpretation consistent with the description as a whole.

The embodiments of the invention may be executed by a computer processor or similar device programmed in the manner of method steps, or may be executed by an electronic system which is provided with means for executing these steps. Similarly, an electronic memory means such as computer diskettes, CD-ROMs, Random Access Memory (RAM), or similar computer software storage media known in the art, may be programmed to execute such method steps. As well, electronic signals representing these method steps may also be transmitted via a communication network.

Embodiments of the invention may be implemented in any conventional computer programming language. For example, preferred embodiments may be implemented in a procedural programming language (e.g.“C”) or an object-oriented language (e.g.“C++”, “java”, “PHP”, “PYTHON” or “C#”) or in any other suitable programming language (e.g. “Machine code”, “Assembly”, “Go”, “Dart”, “Ada”, “Bash”, etc.). Alternative embodiments of the invention may be implemented as pre-programmed hardware elements, other related components, or as a combination of hardware and software components.

Embodiments can be implemented as a computer program product for use with a computer system. Such implementations may include a series of computer instructions fixed either on a tangible medium, such as a computer readable medium (e.g., a diskette, CD-ROM, or fixed disk) or transmittable to a computer system, via a modem or other interface device, such as a communications adapter connected to a network over a medium. The medium may be either a tangible medium (e.g., optical or electrical communications lines) or a medium implemented with wireless techniques (e.g., microwave, infrared or other transmission techniques). The series of computer instructions embodies all or part of the functionality previously described herein. Those skilled in the art should appreciate that such computer instructions can be written in a number of programming languages for use with many computer architectures or operating systems. Furthermore, such instructions may be stored in any memory device, such as semiconductor, magnetic, optical or other memory devices, and may be transmitted using any communications technology, such as optical, infrared, microwave, or other transmission technologies. It is expected that such a computer program product may be distributed as a removable medium with accompanying printed or electronic documentation (e.g., shrink-wrapped software), preloaded with a computer system (e.g., on system ROM or fixed disk), or distributed from a server over a network (e.g., the Internet or World Wide Web). Of course, some embodiments of the invention may be implemented as a combination of both software (e.g., a computer program product) and hardware. Still other embodiments of the invention may be implemented as entirely hardware, or entirely software (e.g., a computer program product).

A person understanding this invention may now conceive of alternative structures and embodiments or variations of the above all of which are intended to fall within the scope of the invention as defined in the claims that follow.

We claim:

1. An electrode comprising:
at least one section comprising modified graphite;
wherein
said electrode is for use in measuring a level of chlorine in a liquid sample;
said modified graphite is modified by a process comprising:
immersing graphite in an electrolyte solution with said graphite operating as a working electrode; and
applying a voltage to said graphite such that there is a voltage potential difference between said working electrode and a reference electrode of at least 0.8 V, and, at most, 1.15 V;
wherein
said electrolyte comprises ammonium carbamate prepared in a sodium phosphate buffer.

2. The electrode according to claim 1, wherein the voltage potential difference is at least 1.1 V and is, at most, 1.15 V.

3. The electrode according to claim 1, wherein said measuring the level of chlorine is correlated according to an equation:
i=kct^{-0.5}+bc

4. A process for modifying graphite, the process comprising:
immersing said graphite in an electrolyte solution with said graphite operating as a working electrode; and
applying a voltage to said graphite such that there is a voltage potential difference between said working electrode and a reference electrode, said voltage difference being at least 0.8 V and, at most, 1.15 V;
wherein a resulting modified graphite is used in an electrode for measuring chlorine in a liquid sample.

5. The process according to claim 4, wherein the voltage potential difference is at least 1.1 V and, at most, 1.15 V.

6. The process according to claim 4, wherein said electrode is for use in measuring an amount of chlorine in a static liquid sample.

7. A method for measuring a level of chlorine, the method comprising:
a) providing a sensor system in a liquid sample, said sensor system comprises a working electrode, a reference electrode and a counter electrode, said working electrode comprising:
at least one section comprising modified graphite; wherein
said modified graphite is modified by a process comprising:
immersing graphite in an electrolyte solution; and
applying a voltage to said graphite such that there is a voltage potential difference between said graphite and a modification reference electrode of at least 0.8 V, and, at most, 1.15 V; and wherein
said electrolyte comprises ammonium carbamate prepared in a sodium phosphate buffer;
b) applying a constant voltage between the working electrode and the reference electrode;
c) measuring a current of the working electrode over time during application of the constant voltage;
d) correlating the current to the level of chlorine in the liquid sample.

8. The method according to claim 7, wherein the constant voltage between the working electrode and the reference electrode in step b) is 0.1 V.

9. The method according to claim 7, wherein the counter electrode is an unmodified graphite electrode and the reference electrode is Ag/AgCl.

10. The method according to claim 7, wherein the correlating step comprises calculations including an equation:
i=kct^{-0.5}+bc

11. A method for measuring a level of chlorine, the method comprising:
a) providing a sensor system in a liquid sample, said sensor system comprises a working electrode and a reference electrode, said working electrode comprising:
at least one section comprising modified graphite; wherein
said modified graphite is modified by a process comprising:
immersing graphite in an electrolyte solution; and
applying a voltage to said graphite such that there is a voltage potential difference between said graphite and a modification reference electrode of at least 0.8 V, and, at most, 1.15 V; and wherein
said electrolyte comprises ammonium carbamate prepared in a sodium phosphate buffer;
b) applying a voltage between the working electrode and the reference electrode in the form of a plurality of pulsations of a fixed duration, and at a fixed interval between each pair of said plurality of pulsations;
c) measuring a current of the working electrode at an end of each of said plurality of pulsations;
d) correlating the current to the level of chlorine in the liquid sample.

12. The method according to claim 11, wherein the voltage of each of the plurality of pulsations is 0.1 V.

13. The method according to claim 11, wherein the fixed duration of each of the plurality of pulsations is at least 15 ms and, at most 60,000 ms.

14. The method according to claim 11, wherein the fixed duration of each of the plurality of pulsations is 30 ms.

15. The method according to claim 11, wherein the fixed interval is from 120 ms to 120,000 ms between each of said plurality of pulsations.

16. The method according to claim 11, wherein the fixed interval is 500 ms between each of said plurality of pulsations.

17. The method according to claim 11, wherein the method further comprises measuring the conductivity value of the liquid sample for calculations used in step d).

18. The method according to claim 11, wherein the conductivity value is measured using a least one of a separate conductivity meter and an integrated conductivity meter.

19. A method according to claim 11, wherein the level of chlorine is from 0 ppm to 20 ppm.

20. The method according to claim 11, wherein the level of chlorine is from 0 ppm to 5 ppm.

21. The method according to claim 11, wherein the reference electrode is an unmodified graphite electrode.

22. The method according to claim 11, wherein the reference electrode is in the form of two separate electrodes comprising a reference electrode and a counter electrode.
